(19) United States
(12) Patent Application Publication
(10) Pub. No.: US 2009/0258929 A1
(43) Pub. Date: Oct. 15, 2009

(54) Compositions and Methods for Modulating mTOR Signaling

(76) Inventors: Peter Finan, Marblehead, MA (US); Jeff Mac Keigan, East Rapids, MI (US); Leon Murphy, Brookline, MA (US); Paul Leslie Nicklin, West Sussex (GB); Ellen Triantafellow, Brighton, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/439,621

(22) PCT Filed: Aug. 28, 2007
(86) PCT No.: PCT/US07/76934

§ 371 Date: Mar. 2, 2009

Related U.S. Application Data
(60) Provisional application No. 60/823,972, filed on Aug. 30, 2006.

(51) Int. Cl. A61K 31/7088; C12Q 1/68; A61P 35/00

(52) U.S. Cl. 514/44 A; 435/6

(57) ABSTRACT

The present invention relates to the use of antagonists of the mTOR signaling pathway and its constituent members (e.g., antagonists of MAPKAP) for the treatment, amelioration, and diagnosis of PI3K/AKT/mTOR-related disorders, e.g., cancers. The present invention also relates to methods of using modulators of mTOR (e.g., modulators of MAPKAP) for the treatment, amelioration, and diagnosis of PI3K/AKT/mTOR-related disorders, e.g., cancers. Assays for the identification of modulators of mTOR, MAPKAP, and hVPS34 are also provided.

### BACKGROUND OF THE INVENTION

Cell growth can be caused by a variety of factors, including nutrient availability, growth factor (e.g., insulin, IGF) availability, and the cell's energy status. The mTOR (mammalian Target Of Rapamycin) signaling pathway integrates all three inputs to control cell growth, and is capable of exerting activity as both an intra- and inter-cellular signaling mechanism. That is, TOR controls not only the growth of cells in which it resides, but also affects the growth of distantly-located cells, thereby possessing the ability to affect overall organ (and organism) size.

mTOR1 and mTOR2 are the mammalian orthologs of a pair of serine/threonine kinases originally identified in yeast (Saccharomyces cerevisiae). mTORC1 (the complex of mTOR, mLST8 (also known as GβL), and RAPTOR (mammalian ortholog of KOG1, or “Kontroller Of Growth-1”)) is structurally conserved relative to the yeast version, and is associated with the modulation of cell growth through regulating translation, transcription, nutrient transport, ribosome biogenesis, and autophagy in response to nutrient availability (Martin et al. (2005) Curr. Opinion in Cell Bio. 17: 158).

mLST8/GβL, RAPTOR, and mTOR comprise nutrient-sensitive complex mTORC1, and mLST8/βL regulates the stability of mTOR-RAPTOR under different nutrient conditions; GβL binds to the kinase domain of mTOR and stabilizes the interaction of RAPTOR with mTOR (Kim et al. (2003) Mol. Cell. 11(4):895). RAPTOR is a positive regulator of mTOR, and plays roles in nutrient-stimulated signaling to the downstream effector S6K1 (S6 Kinase 1), maintenance of cell size, and mTOR protein expression. Conditions that repress the pathway, such as nutrient deprivation and mitochondrial uncoupling, stabilize the mTOR-RAPTOR association and inhibit mTOR kinase activity.

Phosphoinositide 3-kinases (hereafter, “PI3Ks”) are enzymes that phosphorylate the 3-hydroxyl position of the inositol ring of phosphoinositides (“PIs”), and are involved in diverse cellular events such as cell migration, cell proliferation, oncogenic transformation, cell survival, signal transduction, and intracellular trafficking of proteins. In yeast, the VPS34 gene product is a PI3-kinase that mediates the active diversion of proteins from the secretory pathway to vacuoles, and mammals have a corresponding family of PI3-kinases, including three classes of PI3Ks, with a variety of isoforms and types within. The closest human homolog of yeast VPS34 is an 887 residue protein called PI3Kclass3 (“PI3KC3”) (also referred to herein as “hVPS34,” for human VPS34), which shares about 37% sequence identity with the yeast protein over its full length (Volinia et al. (1995) EMBO J. 14(14): 3339).

The nutrient-sensitive TOR pathway and the IGF-PI3K (growth factor) signaling pathway are functionally connected, and are known to crosstalk. mTOR is a downstream mediator in the PI3K/AKT signaling pathway; mitogens activate the mTOR pathway through phosphatidylinositol 3-kinase (PI3K). In certain disease states, the activated mTOR pathway is part of a pathological signaling cascade, e.g., one implicated in the abnormal growth and survival of certain cancer cells.

However, an understanding of the way in which these pathways interact with one another represents an unmet need, and an identification of shared binders would help advance the treatment of, for example, disorders associated with mTOR signaling (e.g., with nutrient-induced mTOR signaling). Furthermore, discovery of methods of modulating the PI3K/AKT/mTOR pathway, e.g., by modulating constituent pathway protein members and their binders/interactors, would be useful for diagnosing, ameliorating the symptoms of, protecting against, and treating PI3K/AKT/mTOR-related disorders and other related malignancies.

### SUMMARY OF THE INVENTION

The present invention relates to diagnosing, ameliorating the symptoms of, protecting against, and treating disorders related to aberrant PI3K/AKT/mTOR signaling (e.g., cancers and other related malignancies), e.g., through use of mTOR antagonists. Examples include methods of treating disorders related to aberrant mTOR signaling (e.g., cancers and other related malignancies) through the administration of inhibitors of MAPKAP, such as siRNA inhibitors of MAPKAP. Aberrant PI3K/AKT/mTOR signaling can be manifested as the overexpression of nutrient transporters, for instance.

The invention also relates to MAPKAP antagonists, including agents capable of preventing MAPKAP (in its different isoforms) from binding to and forming complexes with members of the mTOR/AKT/PI3K pathway (including their substituent pathway members). Examples of MAPKAP antagonists include siRNA inhibitors of MAPKAP.

The invention further relates to methods of identifying and testing agonists and antagonists of mTOR and its associated proteins. The discovery of a protein: protein interaction between MAPKAP, mTOR, and hVPS34 is useful for identifying agents that will enhance or interfere with this binding event (or its resultant complex formation) in vivo or in vitro, and for discovering agents that can be used to treat disorders associated with the absence or presence of this binding event (or its resultant complex formation).

The invention further relates to methods of identifying and testing agonists and antagonists of hVPS34 (PI3KC3) and its associated proteins. The discovery of a protein: protein interaction between MAPKAP, mTOR, and hVPS34 is useful for identifying agents that will enhance or interfere with this binding event (or its resultant complex formation) in vivo or vitro, and for discovering agents that can be used to treat disorders associated with the absence or presence of this binding event (or its resultant complex formation).

The present invention includes a method for screening compounds or other agents for treating PI3K/AKT/mTOR related disorders, comprising: a) providing i) a mTOR complex member protein or homolog capable of binding to a MAPKAP protein; ii) a MAPKAP protein or homolog known to interact with said mTOR complex member; and iii) one or more test agents for screening; b) mixing, in any order, said mTOR complex member protein or homolog, said MAPKAP protein or homolog, and said one or more compound to be tested; and c) measuring the alteration of the mTOR complex member protein (or analog): MAPKAP (or analog) binding in the presence of the test agent, as compared to the binding in absence of said compound.

The present invention includes a method for screening compounds or other agents for treating PI3K/AKT/mTOR related disorders, comprising: a) providing i) a PI3K complex member protein or homolog (e.g., hVPS34) capable of binding to a MAPKAP protein; ii) a MAPKAP protein or homolog known to interact with said PI3K complex member; and iii) one or more test agents for screening; b) mixing, in any order, said PI3K complex member protein or homolog (e.g., hVPS34), said MAPKAP protein or homolog, and said one or more compound to be tested; and c) measuring the alteration of the PI3K complex member protein (or analog): MAPKAP (or analog) binding in the presence of the test agent, as compared to the binding in absence of said compound.

### BRIEF DESCRIPTION OF THE FIGURES

FIG. 1 shows MAPKAP RNA levels (two isoforms of MAPKAP) in HeLa cells transfected with different anti-MAPKAP siRNAs, as measured by quantitative RT-PCR. The cells were transfected with the siRNAs and controls (untransfected cells and scrambled control cells) for 30 hours, and then processed for TaqMan, as described herein.

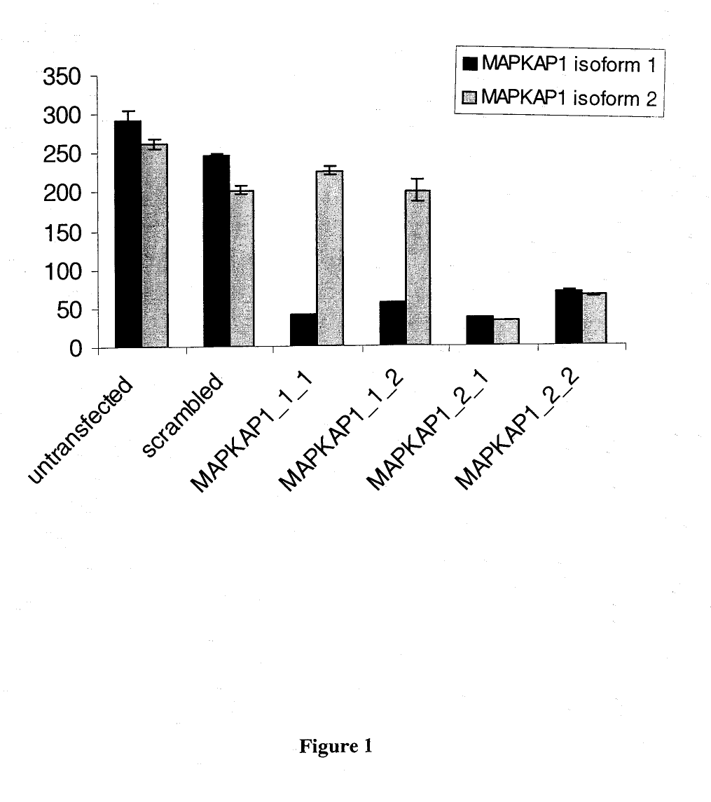

FIG. 2 shows a Western blot that demonstrates the presence or absence of proteins in the PI3K/mTOR pathway, in HeLa cells transfected with a combination of anti-MAPKAP siRNA sequences, nutrients, and insulin. The blot demonstrates that anti-MAPKAP siRNA sequences are capable of inhibiting nutrient-induced, but not insulin-induced, mTOR signaling.

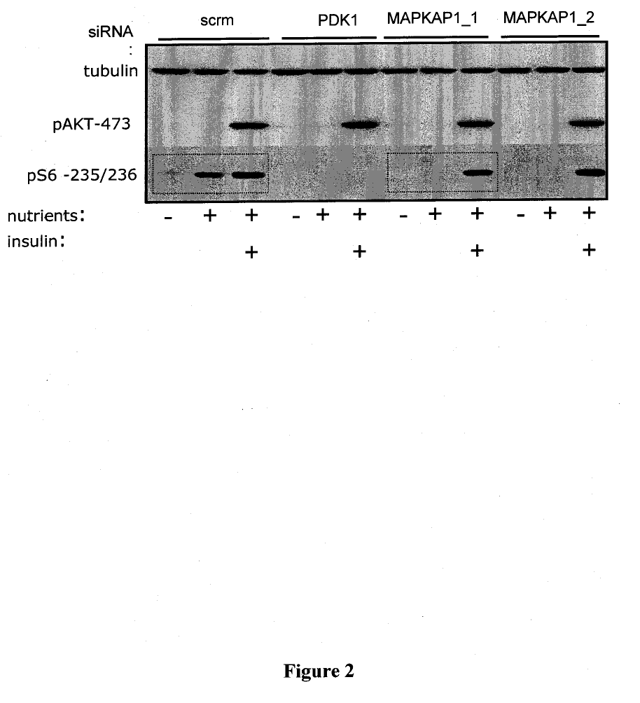

### DETAILED DESCRIPTION OF THE INVENTION

It is contemplated that the invention described herein is not limited to the particular methodology, protocols, and reagents described, as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication which might be used in connection with the invention.

In practicing the present invention, many conventional techniques in molecular biology are used. These techniques are well known and are explained in, for example, Harlow, E. and Lane, eds., 1988, “Antibodies: A Laboratory Manual”, Cold Spring Harbor Press, Cold Spring Harbor, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

In the present description, the term “treatment” includes both prophylactic or preventive treatment as well as curative or disease suppressive treatment, including treatment of patients at risk of apoptotic-related disorders as well as ill patients. This term further includes the treatment for the delay of progression of the disease.

By “suppress and/or reverse,” e.g., a disorder associated with aberrant PI3K/AKT/mTOR-related signaling in a patient (e.g., a cancer), Applicants mean to abrogate said condition, or to render said condition less severe than before or without the treatment.

As used herein, “modulate” indicates the ability to control or influence directly or indirectly, and by way of non-limiting examples, can alternatively mean inhibit or stimulate, agonize or antagonize, hinder or promote, and strengthen or weaken.

“Cure” as used herein means to lead to the remission of the disorder associated with aberrant PI3K/AKT/mTOR-related signaling in a patient, or of ongoing episodes thereof, through treatment.

The terms “prophylaxis” or “prevention” mean impeding the onset or recurrence of PI3K/AKT/mTOR-related disorders, e.g., cancers.

“Disorders related to aberrant PI3K/AKT/mTOR signaling (e.g., cancers and other related malignancies),” “PI3K/AKT/mTOR signaling-related disorders,” “PI3K/AKT/mTOR related disorders,” “aberrant PI3K/AKT/mTOR signaling-related disorders,” and all similarly used terms include but are not limited to cancers, transplantation-related disorders (e.g., lowering rejection rates, graft-versus-host disease, etc.), muscular sclerosis (MS), arthritis, allergic encephalomyelitis, and other immunosuppressive-related disorders, metabolic disorders (e.g., diabetes), reducing intimal thickening following vascular injury, and misfolded protein disorders (e.g., Alzheimer's Disease, Gaucher's Disease, Parkinson's Disease, Huntington's Disease, cystic fibrosis, macular degeneration, retinitis pigmentosa, and prion disorders) (as mTOR inhibition can alleviate the effects of misfolded protein aggregates). A non-limiting list of the cancers associated with pathological mTOR signaling cascades includes breast cancer, renal cell carcinoma, and prostate cancer. The list also includes cancers and tumors that are associated with the overexpression of amino acid transporters (e.g., LAT1), or which harbor mutations in PTEN, TSC1/2, or PI3KclassA.

The list of “disorders related to aberrant PI3K/AKT/mTOR signaling” also includes hamartoma syndromes, such as tuberous sclerosis and Cowden Disease (also termed Cowden syndrome and multiple hamartoma syndrome). Hamartomas are focal malformations resembling a neoplasm, composed of an overgrowth of mature cells and tissues that normally occur in the affected area.

The list of “disorders related to aberrant PI3K/AKT/mTOR signaling” also includes genetic muscle disorders and myopathies, such as human myotubular myopathy. This disorder is characterized by a reduction of the ability of the phosphatase myotubularin, a protein tyrosine phosphatase required for muscle cell differentiation, to dephosphorylate phosphatidylinositol 3-phosphate (PI(3)P). Phosphatidylinositol 3-phosphate, as discussed below, is the product of phosphorylation of PI by a phosphatidylinositol 3-kinase (PI3K), and targeting hVPS34 in many genetic muscle disorders and myopathies can therapeutically reduce levels of PI(3)P.

As used herein, the term “cancer” includes solid mammalian tumors as well as hematological malignancies. “Solid mammalian tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin. The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS. In addition, a cancer at any stage of progression can be treated, such as primary, metastatic, and recurrent cancers. Information regarding numerous types of cancer can be found, e.g., from the American Cancer Society, or from, e.g., Wilson et al. (1991) Harrison's Principles of Internal Medicine, 12th Edition, McGraw-Hill, Inc. Both human and veterinary uses are contemplated.

As used herein the terms “normal mammalian cell” and “normal animal cell” are defined as cells that are growing under normal growth control mechanisms (e.g., genetic control) and display normal cellular differentiation. Cancer cells differ from normal cells in their growth patterns and in the nature of their cell surfaces. For example cancer cells tend to grow continuously and chaotically, without regard for their neighbors, among other characteristics well known in the art.

As used herein, the term “inhibitory nucleic acid” refers to nucleic acid compounds capable of producing gene-specific inhibition of gene expression. Typical inhibitory nucleic acids include, but are not limited to, antisense oligonucleotides, triple helix DNA, RNA aptamers, ribozymes and short inhibitory RNAs (“siRNAs”). For example, knowledge of a nucleotide sequence may be used to design siRNA or antisense molecules which potently inhibit the expression of mTOR pathway proteins (including analogs or variants), or that of their binders/interactors. Similarly, ribozymes can be synthesized to recognize specific nucleotide sequences of a gene and cleave it. Techniques for the design of such molecules for use in targeted inhibition of gene expression are well known to one of skill in the art.

“Cure” as used herein means to lead to the remission of the disorder, e.g., the PI3K/AKT/mTOR-related disorder, or of ongoing episodes thereof, through treatment.

The terms “prophylaxis” or “prevention” means impeding the onset or recurrence of PI3K/AKT/mTOR-related disorders, e.g., cancers.

“Delay of progression” as used herein means that the administration of the modulator (e.g., mTOR antagonist) to patients in a pre-stage or in an early phase of a PI3K/AKT/mTOR-related disorder in a patient (e.g., cancer) prevents the disease from evolving further, or slows down the evolution of the disease in comparison to the evolution of the disease without administration of the modulator.

As used herein a “small organic molecule,” or “small molecule,” is an organic compound (or organic compound complexed with an inorganic compound (e.g., metal) that has a molecular weight of less than 3 kilodaltons, and preferably less than 1.5 kilodaltons.

As used herein a “reporter” gene is used interchangeably with the term “marker gene” and is a nucleic acid that is readily detectable and/or encodes a gene product that is readily detectable such as luciferase.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrases “therapeutically effective amount” and “effective amount” are used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host.

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

“Analog” as used herein, refers to a small organic compound, a nucleotide, a protein, or a polypeptide that possesses similar or identical activity or function(s) as the compound, nucleotide, protein or polypeptide or compound having the desired activity and therapeutic effect of the present invention. (e.g., inhibition of tumor growth), but need not necessarily comprise a sequence or structure that is similar or identical to the sequence or structure of the preferred embodiment.

“Derivative” refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide.

“Inhibitors,” or “antagonists” refer to inhibitory molecules identified using in vitro and in vivo assays for PI3K/AKT/mTOR pathway function. Inhibitors and antagonists can refer to agents that decrease signaling that occurs via the PI3K/AKT/mTOR pathway. Inhibitors may be agents that decrease, block, or prevent, signaling via this pathway, and/or which prevent the formation of protein complexes such as the mTORC1 or related complexes.

As used herein, the term “cancer” includes solid mammalian tumors as well as hematological malignancies. “Solid mammalian tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin, central nervous system including brain; sarcomas of the soft tissue and bone; and melanoma of cutaneous and intraocular origin. The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS. In addition, a cancer at any stage of progression can be treated, such as primary, metastatic, and recurrent cancers. Information regarding numerous types of cancer can be found, e.g., from the American Cancer Society, or from, e.g., Wilson et al. (1991) Harrison's Principles of Internal Medicine, 12th Edition, McGraw-Hill, Inc.

“G,” “C,” “A” and “U” each generally stand for a nucleotide that contains guanine, cytosine, adenine, and uracil as a base, respectively. However, it will be understood that the term “ribonucleotide” or “nucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine. Sequences comprising such replacement moieties are embodiments of the invention.

As used herein, “target sequence” refers to a contiguous portion of the nucleotide sequence of an mRNA molecule formed during the transcription of the MAPKAP gene, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a siRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes of the invention.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled.

The terms “complementary”, “fully complementary” and “substantially complementary” herein may be used with respect to the base matching between the sense strand and the antisense strand of a siRNA, or between the antisense strand of a siRNA and a target sequence, as will be understood from the context of their use.

As used herein, a polynucleotide which is “substantially complementary to at least part of” a messenger RNA (mRNA) refers to a polynucleotide which is substantially complementary to a contiguous portion of the mRNA of interest (e.g., encoding MAPKAP). For example, a polynucleotide is complementary to at least a part of a MAPKAP mRNA if the sequence is substantially complementary to a non-interrupted portion of a mRNA encoding MAPKAP.

The term “double-stranded RNA” or “dsRNA”, as used herein, refers to a complex of ribonucleic acid molecules, having a duplex structure comprising two anti-parallel and substantially complementary, as defined above, nucleic acid strands. The two strands forming the duplex structure may be different portions of one larger RNA molecule, or they may be separate RNA molecules. Where separate RNA molecules, such siRNA are often referred to in the literature as siRNA (“short interfering RNA”). Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”, “short hairpin RNA” or “shRNA”. Where the two strands are connected covalently by means other than an uninterrupted chain of nucleotides between the 3′-end of one strand and the 5′ end of the respective other strand forming the duplex structure, the connecting structure is referred to as a “linker”. The RNA strands may have the same or a different number of nucleotides. The maximum number of base pairs is the number of nucleotides in the shortest strand of the siRNA minus any overhangs that are present in the duplex. In addition to the duplex structure, a siRNA may comprise one or more nucleotide overhangs. In addition, as used in this specification, “siRNA” may include chemical modifications to ribonucleotides, including substantial modifications at multiple nucleotides and including all types of modifications disclosed herein or known in the art. Any such modifications, as used in an siRNA type molecule, are encompassed by “siRNA” for the purposes of this specification and claims.

As used herein, a “nucleotide overhang” refers to the unpaired nucleotide or nucleotides that protrude from the duplex structure of a siRNA when a 3′-end of one strand of the siRNA extends beyond the 5′-end of the other strand, or vice versa. “Blunt” or “blunt end” means that there are no unpaired nucleotides at that end of the siRNA, i.e., no nucleotide overhang. A “blunt ended” siRNA is a siRNA that is double-stranded over its entire length, i.e., no nucleotide overhang at either end of the molecule. For clarity, chemical caps or non-nucleotide chemical moieties conjugated to the 3′ end or 5′ end of an siRNA are not considered in determining whether an siRNA has an overhang or is blunt ended.

The term “antisense strand” refers to the strand of a siRNA which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a siRNA that includes a region that is substantially complementary to a region of the antisense strand.

“Introducing into a cell”, when referring to a siRNA, means facilitating uptake or absorption into the cell, as is understood by those skilled in the art. Absorption or uptake of siRNA can occur through unaided diffusive or active cellular processes, or by auxiliary agents or devices. The meaning of this term is not limited to cells in vitro; a siRNA may also be “introduced into a cell”, wherein the cell is part of a living organism. In such instance, introduction into the cell will include the delivery to the organism. For example, for in vivo delivery, siRNA can be injected into a tissue site or administered systemically. In vitro introduction into a cell includes methods known in the art such as electroporation and lipofection.

The terms “silence” and “inhibit the expression of”, in as far as they refer to the MAPKAP gene or any genes involved in the PI3K/AKT/mTOR signaling pathways, herein refer to the at least partial suppression of the expression of the MAPKAP gene (or genes involved in the PI3K/AKT/mTOR signaling pathways), as manifested by a reduction of the amount of mRNA transcribed from the MAPKAP gene (or genes involved in the PI3K/AKT/mTOR signaling pathways) which may be isolated from a first cell or group of cells in which the MAPKAP gene (or genes involved in the PI3K/AKT/mTOR signaling pathways) is transcribed and which has or have been treated such that the expression of the MAPKAP gene (or genes involved in the PI3K/AKT/mTOR signaling pathways) is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to MAPKAP gene (or genes involved in the PI3K/AKT/mTOR signaling pathways) transcription, e.g. the amount of protein encoded by the MAPKAP gene (or genes involved in the PI3K/AKT/mTOR signaling pathways) which is secreted by a cell, or the number of cells displaying a certain phenotype, e.g., uncontrolled proliferation. In principle, MAPKAP gene (or genes involved in the PI3K/AKT/mTOR signaling pathways) silencing may be determined in any cell expressing the target, either constitutively or by genomic engineering, and by any appropriate assay. However, when a reference is needed in order to determine whether a given siRNA inhibits the expression of the MAPKAP gene (or genes involved in the PI3K/AKT/mTOR signaling pathways) by a certain degree and therefore is encompassed by the instant invention, the assay provided in the Examples below shall serve as such reference.

For example, in certain instances, expression of the MAPKAP gene is suppressed by at least about 20%, 25%, 35%, or 50% by administration of the double-stranded oligonucleotide of the invention. In some embodiment, the MAPKAP gene is suppressed by at least about 60%, 70%, or 80% by administration of the double-stranded oligonucleotide of the invention. In some embodiments, the MAPKAP gene is suppressed by at least about 85%, 90%, or 95% by administration of the double-stranded oligonucleotide of the invention.

mTOR

mTOR (mammalian target of rapamycin) is a large serine/threonine kinase originally identified as TOR in yeast (Saccharomyces cerevisiae), and discovered during a screen for resistance to the immunosuppressant rapamycin (also known by its USAN generic name, sirolimus) (see, e.g., Kunz et al. (1993) Cell (73): 585, or U.S. Pat. No. 3,929,992). It is a member of the PI3K (phosphoinositide 3-kinases) family of protein kinases, identified by homology within its catalytic domain.

In yeast and mammals, the identification of two structurally and functionally distinct multiprotein TOR complexes (TORC1 and TORC2) has provided a molecular basis for the complexity of TOR signaling. mTOR activity is regulated by at least three upstream inputs: amino acids, glucose, and growth factors. In addition to regulating cell growth via both intra- and inter-cellular signaling mechanisms, TOR has emerged as a regulator of growth-related processes such as development, aging and the response to hypoxia. Thus, TOR is part of an signaling network with a remarkably broad role in eukaryotic biology.

mTORC1 (the complex, or signaling network, of mTOR1) is structurally conserved relative to the yeast version, and is associated with the modulation of cell growth through regulating translation, transcription, nutrient transport, ribosome biogenesis, and autophagy in response to nutrient availability (Martin et al. (2005) Curr. Opinion in Cell Bio. 17: 158). These functions are often referred to as elements of “temporal” control of cell growth, compared to the “spatial” control associated with mTORC2 (that is, regulation of cell growth via modulating the cell-cycle-dependent polarization of the actin cytoskeleton).

mTOR senses, and is activated by, nutrient availability in several ways, such as through the formation of complexes (mTORC1) with pathway proteins mLST8/βL and with RAPTOR (mammalian ortholog of KOG1, or “Kontroller Of Growth-1”). Amino acids, particularly leucine, regulate the formation of nutrient-sensing mTORC1 that facilitates the recognition of substrates by mTOR under nutrient-replete conditions. Other downstream mTORC1 targets and effectors include ribosomal protein p70 S6 kinase (S6K1), and initiation factor 4E-binding protein-1 (4E-BP1). Activated mTOR kinase phosphorylates/activates S6K1 (at Thr389), and phosphorylates/inactivates 4E-BP1, resulting in the initiation of translation and cell cycle progression. S6K1 regulates protein synthesis, cell size, and proliferation in response to cellular nutritional status and hormonal stimulation (Kozma et al. (2002) Bioessays 24: 65), involving not only mTOR, but the p85/p110 PI3-kinases and the downstream kinases PDK-1 and AKT as well.

mTORC1 (mLST8/βL, RAPTOR, and mTOR) comprises a nutrient-sensitive complex, and mLST8/βL regulates the stability of mTOR-RAPTOR under different nutrient conditions (Kim et al. (2003) Mol. Cell. 11(4):895). RAPTOR is a positive regulator of mTOR, and plays a role in nutrient-stimulated signaling to the downstream effector S6K1, maintenance of cell size, and mTOR protein expression. Conditions that repress the pathway, such as nutrient deprivation and mitochondrial uncoupling, stabilize the mTOR-RAPTOR association and inhibit mTOR kinase activity.

GβL binds to the kinase domain of mTOR and stabilizes the interaction of RAPTOR with mTOR. Like mTOR and RAPTOR, GβL participates in nutrient- and growth factor-mediated signaling to S6K1, a downstream effector of mTOR, and in the control of cell size. The binding of GβL to mTOR strongly stimulates the kinase activity of mTOR toward S6K1 and 4E-BP1, an effect reversed by the stable interaction of RAPTOR with mTOR. Interestingly, nutrients and rapamycin regulate the association between mTOR and RAPTOR only in complexes that also contain GβL.

In addition to its role as nutritional status checkpoint, mTORC1 is an important signaling intermediate molecule downstream of the PI3K/AKT pathway (e.g., see Grunwald et al. (2002) Cancer Res. 62: 6141; and Stolovich et al. (2002) Mol Cell Biol. 22: 8101). The discovery of other upstream regulators of TOR (e.g., AMPK, the TSC1-TSC2 (tuberous sclerosis complex) heterodimer, and Rheb) has provided new insights into the mechanism by which TOR integrates its various inputs. AKT-mediated phosphorylation inhibits the GAP activity of TSC1/TSC2 toward the Rheb GTPase, leading to Rheb activation. Rheb binds directly to mTOR, a process that is regulated by amino acids. Both amino acids and Rheb activation are required for mTOR signaling to S6K1, and the role played by TSC1/TSC2 during amino acid regulation of mTOR is less clear.

### PI3K

Phosphoinositide 3-kinases (hereafter, “PI3Ks”) are enzymes that phosphorylate the 3-hydroxyl position of the inositol ring of phosphoinositides (“PIs”), and are involved in diverse cellular events such as cell migration, cell proliferation, oncogenic transformation, cell survival, signal transduction, and intracellular trafficking of proteins. In yeast, the VPS34 gene product is a PI 3-kinase that mediates the active diversion of proteins from the secretory pathway to vacuoles, and mammals have a corresponding family of PI3-kinases, including three classes of PI3 Ks, with a variety of isoforms and types within. The human homolog of yeast VPS34 is an 887 residue protein called PI3Kclass3 (“PI3KC3”), which shares about 37% sequence identity with the yeast protein over its full length (Volinia et al., (1995) EMBO J. 14(14): 3339).

PI3Ks activate intracellular signaling molecules upon growth factors binding their cell surface receptors. These lipid kinases phosphorylate the inositol ring of phosphatidylinositol and related compounds at the 3-prime position; they are capable of phosphorylating both phosphatidylinositols (also referred to as “PtdIns”) and phosphoinositides (“PIs”, which are phosphorylated versions of phosphatidylinositols).

The products of these reactions serve as secondary messengers in growth signaling pathways, influencing such cellular events as cell survival, migration, motility, and proliferation; oncogenic transformation; tissue neovascularization; and intracellular protein trafficking. Cell-surface receptors induce the production of second messengers such as phosphatidylinositol 4,5-bisphosphate 3, which convey signals from the cell surface to cytoplasm. These secondary messengers activate the PI3K-dependent protein kinase-1, which in turn activates the kinase AKT. AKT activation leads to phosphorylation of proteins leading to cell survival. (Cantley et al. (1999) PNAS 96:4240).

By way of example, AKT phosphorylates IκB, thereby activating NFκcB and promoting cellular survival. AKT also phosphorylates Bad (a proapoptotic Bcl-2 family member) and caspase 9, in both cases blocking the induction of apoptosis. Other downstream targets of AKT include forkhead-related transcription factor 1 and mammalian target of rapamycin (mTOR).

PI3Ks are grouped in three classes, categorized according to their structure, substrate specificity, physiological function, and tissue-specificity. (Domin et al. (1997) FEBS Lett. 410:91). Class I PI3Ks are mostly cytosolic, are heterodimers comprising a p110 catalytic subunit and an adaptor/regulatory subunit, and are further divided into two classes: Class IA PI3Ks consist of a p110 catalytic subunit that associates with an SH2 domain-containing subunit p85, and is activated by the majority of tyrosine kinase-coupled transmembrane receptors; class IB PI3K consists of a p101 regulatory subunit that associates with p110γ catalytic subunit, and is activated by heterotrimeric G-protein-coupled-receptors. (Katso et al. (2001) Annu. Rev. Cell Dev. Biol. 17:615). Class II PI3Ks consist of three isoforms, as discussed herein. Class III PI3Ks utilize only phosphatidylinositol as a substrate, and play an essential role in protein trafficking through the lysosome. (Volinia, et al. (1995) EMBO J. 14:3339).

Phosphatidylinositol 3-phosphate (PI3P) is the product of phosphorylation of PI by a phosphatidylinositol 3-kinase (PI3K). Although the major source of PI3P in most eukaryotic cells is PI3KC3, all PI3Ks can generate this lipid in vitro. Proteins with FYVE domains, many of which are involved in vesicle trafficking, directly bind to the head group of PI3P as a mechanism of localization at specific membranes.

### VPS34

The VPS34 gene product (VPS34p) is an enzyme required for protein sorting to the lysosome-like vacuole of the yeast, and appears to regulate intracellular protein trafficking decisions. VPS34p shares significant sequence similarity with the catalytic subunit of bovine phosphatidylinositol (PI) 3-kinase (the p110 subunit), which is known to interact with activated cell surface receptor tyrosine kinases. Yeast strains deleted for the VPS34 gene or carrying VPS34 point mutations lacked detectable PI 3-kinase activity and exhibited severe defects in vacuolar protein sorting. Overexpression of VPS34p resulted in an increase in PI 3-kinase activity, and this activity was specifically precipitated with antisera to VPS34p (Schu et al. (1993) Science 260 (5104): 88).

The third class of PI3 kinase, PI3KC3, is related to the S. cerevisiae gene VPS34. This human homologue of VPS34 is complexed with a ser/thr kinase called VPS15p. Of the three classes of PI3 kinase this has the most restricted substrate specificity, being strictly limited to PtdIns. Like class IA PI3-kinases, PI3KC3s (e.g., hVPS34) play a well recognized role in the regulation of S6K1, and hence in nutrient-sensing.

hVPS34 is not part of the insulin input to S6K1 (i.e., is not stimulated by insulin). However, hVPS34 is inhibited by amino acid or glucose starvation, suggesting that it lies on the nutrient-regulated pathway to S6K1. Consistent with this, hVPS34 is also inhibited by activation of the AMP-activated kinase, which inhibits mTOR/S6K1 in glucose-starved cells. hVPS34 appears to lie upstream of mTOR, as small interfering RNA knock-down of hVPS34 inhibits the phosphorylation of mTOR substrate 4EBP1. hVPS34 is though to be a nutrient-regulated lipid kinase that integrates amino acid and glucose inputs to mTOR and S6K1.

MAPKAP

MAPKAP (GenBank accession number NM_{—}024117), also known as human SIN1, is a highly conserved ortholog of the SIN1 family, members of which are widely distributed in the fungal and metazoan kingdoms. Study of the non-human orthologs of MAPKAP has suggested a role for this gene and its protein product(s) in intracellular signaling interactions with Ras, mTOR, and the SAPKs (stress-activated protein kinases). MAPKAP transcripts can use alternative polyadenylation signals and describe a number of MAPKAP splice variants that potentially encode functionally different isoforms (Schroder et al. (2004) Gene 339: 17). MAPKAP transcript variant 2 (SIN1β) differs from the full length transcript (variant 1) by a deletion of 36 amino acids corresponding to residues P321-S356 in the protein encoded by transcript variant 1. A total of six MAPKAP transcript variants have been reported, although very little is known about functional differences between the variants. Immunoblot analysis suggests that the proteins encoded by the splicing variants are differentially expressed in different cell lines.

It is not clear if any functional differences result from the alternative splicing or from tissue-specific expression. One group (Schroder et al. (2005) Cellular Signaling 17) has shown that the stress activated protein kinase JNK co-immunoprecipitates with exogenously expressed proteins encoded by transcript variant 1 and transcript variant 6 (which contains a C-terminal truncation of 202 amino acids followed by an additional ACD sequence). The same group has demonstrated that expression of full length MAPKAP protein appears to partially inhibit the activation of JNK kinase following UV-irradiation of cells.

As the data described in the present application demonstrates, MAPKAP is a binding partner of both mTOR and hVPS34, providing a vital link between those pathways and helping to elucidate the linkage and crosstalk between the PI3K/AKT/mTOR signaling pathways. Furthermore, the data described herein shows the actual modulation of these pathways, by disrupting binding events involving MAPKAP.

The invention relates to the methods and compositions for treatment, diagnosis, and/or amelioration of PI3K/AKT/mTOR-related disorders, e.g., cancers. In one embodiment, the invention relates to the use of inhibitory nucleic acids to treat PI3K/AKT/mTOR-related disorders, e.g., cancers.

The present invention relates to diagnosing, ameliorating the symptoms of, protecting against, and treating disorders related to aberrant PI3K/AKT/mTOR signaling (e.g., cancers and other related malignancies), e.g., through use of mTOR antagonists. Examples include methods of treating disorders related to aberrant mTOR signaling (e.g., cancers and other related malignancies) through the administration of inhibitors of MAPKAP, such as siRNA inhibitors of MAPKAP.

The invention further relates to methods of identifying and testing agonists and antagonists of mTOR and its associated proteins. The discovery of a protein: protein interaction between MAPKAP, mTOR, and hVPS34 is useful for identifying agents that will enhance or interfere with this binding event (or its resultant complex formation) in vivo, and for discovering agents that can be used to treat disorders associated with the absence or presence of this binding event (or its resultant complex formation).

The invention further relates to methods of identifying and testing agonists and antagonists of hVPS34 (PI3KC3) and its associated proteins. The discovery of a protein: protein interaction between MAPKAP, mTOR, and hVPS34 is useful for identifying agents that will enhance or interfere with this binding event (or its resultant complex formation) in vivo, and for discovering agents that can be used to treat disorders associated with the absence or presence of this binding event (or its resultant complex formation).

MAPKAP Antagonists

The effects of the mTOR/AKT/PI3K pathway activation in the presence of nutrients are mediated by certain binding events, including but not limited to the binding of MAPKAP to mTOR, the binding of MAPKAP to hVPS34, and the formation of the mTORC1 complex. MAPKAP antagonists include agents capable of preventing MAPKAP (in its different isoforms) from binding to and forming complexes with members of the mTOR/AKT/PI3K pathway (including their substituent pathway members). Examples of MAPKAP antagonists include siRNA inhibitors of MAPKAP. Agents that bind MAPKAP and prevent it from binding to a partner (e.g., in the mTOR/AKT/PI3K pathway, such as mTOR or hVPS34) will act as MAPKAP antagonists.

Screening Assays

The invention provides methods (also referred to herein as “screening assays”) for identifying modulators, i.e., candidate or test compounds or agents (e.g., peptides, peptidomimetics, small molecules or other drugs) which bind to MAPKAP proteins, or to protein members of related pathways or complexes (e.g., the mTOR/AKT/PI3K pathway) and have a stimulatory or inhibitory effect on, for example, MAPKAP expression or activity, or mTOR/AKT/PI3K pathway signaling.

The present invention includes a method for compound or agent screening, comprising: a) providing i) a mTOR complex member protein or homolog capable of binding to a MAPKAP protein; ii) a MAPKAP protein or homolog known to interact with said mTOR complex member; and iii) one or more test agents for screening; b) mixing, in any order, said mTOR complex member protein or homolog, said MAPKAP protein or homolog, and said one or more compound to be tested; and c) measuring the alteration of the mTOR complex member protein (or analog): MAPKAP (or analog) binding in the presence of the test agent, as compared to the binding in absence of said compound.

The present invention includes a method for compound or agent screening, comprising: a) providing i) a PI3K complex member protein or homolog (e.g., hVPS34) capable of binding to a MAPKAP protein; ii) a MAPKAP protein or homolog known to interact with said PI3K complex member; and iii) one or more test agents for screening; b) mixing, in any order, said PI3K complex member protein or homolog (e.g., hVPS34), said MAPKAP protein or homolog, and said one or more compound to be tested; and c) measuring the alteration of the PI3K complex member protein (or analog): MAPKAP (or analog) binding in the presence of the test agent, as compared to the binding in absence of said compound.

In one embodiment, the invention provides assays for screening candidate or test agents which bind a MAPKAP protein or polypeptide or biologically active portion thereof. In another embodiment, the invention provides assays for screening candidate or test agents which bind to or modulate the activity of a MAPKAP protein or polypeptide or biologically active portion thereof, e.g., modulate the ability of MAPKAP to bind to and/or form a complex with other proteins it is normally associated with (e.g., mTOR, hVPS34).

The test agents of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al. (1997) Anticancer Drug Des. 12: 145).

Examples of methods for the synthesis of molecular libraries can be found in the art, for example in: DeWitt et al. (1993) Proc. Natl. Acad. Sci. U.S.A. 90: 6909; Erb et al. (11994) Proc. Natl. Acad. Sci. USA 91: 11422; Zuckermann et al. (1994). J. Med. Chem. 37: 2678; Cho et al. (1993) Science 261: 1303; Carrell et al. (1994) Angew. Chem. Int. Ed. Engl. 33: 2059; Carell et al. (1994) Angew. Chem. Int. Ed. Engl. 33: 2061; and in Gallop et al. (1994) J. Med. Chem. 37: 1233.

Libraries of compounds may be presented in solution (e.g., Houghten (1992) Biotechniques 13: 412), or on beads (Lam (1991) Nature 354: 82), chips (Fodor (1993) Nature 364: 555), bacteria (Ladner, U.S. Pat. No. 5,223,409), spores (Ladner '409), plasmids (Cull et al. (1992) Proc Natl Acad Sci USA 89: 1865) or on phage (Scott and Smith (1990) Science 249: 386); (Devlin (1990) Science 249: 404); (Cwirla et al. (1990) Proc. Natl. Acad. Sci. 87: 6378); (Felici (1991) J. Mol. Biol. 222: 301); (Ladner, supra).

In one embodiment, an assay is a cell-based assay comprising contacting a cell expressing MAPKAP with a test agent, e.g., a test MAPKAP antagonist, and determining the ability of the test agent to modulate (e.g. stimulate or inhibit) the activity of MAPKAP. Determining the ability of the test agent to modulate the activity of an MAPKAP can be accomplished, for example, by determining the ability of the MAPKAP antagonists to bind to MAPKAP or by determining the ability of the MAPKAP antagonists to disrupt the binding event between MAPKAP and mTOR or hVPS34.

Determining the ability of test MAPKAP antagonists to modulate a MAPKAP protein, can be accomplished by determining direct binding. These determinations can be accomplished, for example, by coupling the MAPKAP protein with a radioisotope or enzymatic label such that binding of the protein to a test agent (e.g., a test MAPKAP antagonist) can be determined by detecting the labeled protein in a complex. For example, molecules, e.g., proteins, can be labeled with ¹²⁵I, ³⁵S, ¹⁴C, or ³H, either directly or indirectly, and the radioisotope detected by direct counting of radioemmission or by scintillation counting. Alternatively, molecules can be enzymatically labeled with, for example, horseradish peroxidase, alkaline phosphatase, or luciferase, and the enzymatic label detected by determination of conversion of an appropriate substrate to product.

It is also within the scope of this invention to determine the ability of test MAPKAP antagonists to modulate a MAPKAP protein, or to determine the ability of test agents to modulate proteins that bind to MAPKAP, such as mTOR and hVPS34, without the labeling of any of the interactants. For example, a microphysiometer can be used to detect the interaction of test MAPKAP antagonists with MAPKAP (proteins that bind to MAPKAP, such as mTOR and hVPS34) without the labeling of MAPKAP or the test MAPKAP antagonists (McConnell et al. (1992) Science 257: 1906). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In yet another embodiment, an assay of the present invention is a cell-free assay in which a protein or biologically active portion thereof is contacted with a test agent (e.g., a test MAPKAP antagonist) and the ability of the test agent to bind to the MAPKAP protein, or biologically active portions thereof, is determined. Binding of the test agent to the MAPKAP protein can be determined either directly or indirectly as described above. In a preferred embodiment, the assay includes contacting the MAPKAP protein or biologically active portion thereof with compound known to bind MAPKAP to form an assay mixture, contacting the assay mixture with a test agent, and determining the ability of the test agent to interact with an MAPKAP protein, wherein determining the ability of the test agent to interact with an MAPKAP protein comprises determining the ability of the test agent to preferentially bind to MAPKAP or biologically active portion thereof as compared to the known compound.

In another preferred embodiment, the assay includes contacting MAPKAP proteins or biologically active portion thereof with compound known to bind MAPKAP to form an assay mixture, contacting the assay mixture with a test agent, and determining the ability of the test agent to interact with a MAPKAP protein, wherein determining the ability of the test agent to interact with an MAPKAP protein comprises determining the ability of the test agent to preferentially bind to MAPKAP or biologically active portion thereof as compared to the known compound.

Such a determination may be accomplished using a technology such as real-time Biomolecular Interaction Analysis (BIA). Sjolander et al., 1991 Anal. Chem. 63:2338-2345 and Szabo et al., 1995 Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In more than one embodiment of the above assay methods of the present invention, it may be desirable to immobilize MAPKAP to facilitate separation of complexed from uncomplexed forms of the protein, as well as to accommodate automation of the assay. Binding of a test agent to MAPKAP can be accomplished in any vessel suitable for containing the reactants. Examples of such vessels include microtitre plates, test tubes, and microcentrifuge tubes. In one embodiment, a fusion protein can be provided which adds a domain that allows the protein to be bound to a matrix. For example, glutathione-S-transferase/kinase fusion proteins or glutathione-S-transferase/target fusion proteins can be adsorbed onto glutathione sepharose beads (Sigma Chemical, St. Louis, Mo.) or glutathione derivatized microtitre plates, which are then combined with the test agent or the test agent and the non-adsorbed MAPKAP protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of binding determined using standard techniques.

Other techniques for immobilizing proteins on matrices can also be used in the screening assays of the invention. For example, MAPKAP can be immobilized utilizing conjugation of biotin and streptavidin. Biotinylated MAPKAP protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with MAPKAP proteins or target molecules can be derivatized to the wells of the plate, and unbound MAPKAP protein trapped in the wells by antibody conjugation. Methods for detecting such complexes, in addition to those described above for the GST-immobilized complexes, include immunodetection of complexes using antibodies reactive with the MAPKAP protein or target molecules.

In yet another aspect of the invention, the MAPKAP proteins can be used as “bait proteins” in a two-hybrid assay or three-hybrid assay (see, e.g., U.S. Pat. No. 5,283,317; Zervos et al., 1993 Cell 72:223-232; Madura et al., 1993 J. Biol. Chem. 268:12046-12054; Bartel et al., 1993 Biotechniques 14:920-924; Iwabuchi et al., 1993 Oncogene 8:1693-1696; and Brent WO94/10300), to identify other proteins which bind to MAPKAP. Such MAPKAP binding proteins are also likely to be involved in the propagation of signals by the MAPKAP proteins.

The two-hybrid system is based on the modular nature of most transcription factors, which consist of separable DNA-binding and activation domains. Briefly, the assay utilizes two different DNA constructs. In one construct, the gene that codes for an MAPKAP protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a kinase dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the MAPKAP protein which interacts with the protein.

This invention further pertains to novel agents identified by the above-described screening assays. Accordingly, it is within the scope of this invention to further use an agent identified as described herein in an appropriate animal model. For example, an agent identified as described herein (e.g., an MAPKAP modulating agent) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

RNAi

The invention provides small interfering ribonucleic acid sequences (siRNA), as well as compositions and methods for inhibiting the expression of the MAPKAP gene in a cell or mammal using the siRNA. The invention also provides compositions and methods for treating pathological conditions and diseases in a mammal caused by the aberrant expression of the MAPKAP gene, or caused by the aberrant signaling of pathways of which MAPKAP is an integral member (e.g., PI3K/AKT/mTOR signaling pathway), using siRNA. siRNA directs the sequence-specific degradation of mRNA through a process known as RNA interference (RNAi).

The siRNA of the invention comprises an RNA strand (the antisense strand) having a region which is less than 30 nucleotides in length, generally 19-24 nucleotides in length, and is substantially complementary to at least part of an mRNA transcript of the MAPKAP gene. The use of these siRNAs enables the targeted degradation of mRNAs of genes that are implicated in the PI3K/AKT/mTOR signaling pathway.

The siRNA molecules according to the present invention mediate RNA interference (“RNAi”). The term “RNAi” is well known in the art and is commonly understood to mean the inhibition of one or more target genes in a cell by siRNA with a region which is complementary to the target gene. Various assays are known in the art to test siRNA for its ability to mediate RNAi (see for instance Elbashir et al., Methods 26 (2002), 199-213). The effect of the siRNA according to the present invention on gene expression will typically result in expression of the target gene being inhibited by at least 10%, 33%, 50%, 90%, 95% or 99% when compared to a cell not treated with the RNA molecules according to the present invention.

“siRNA” or “small-interfering ribonucleic acid” according to the invention has the meanings known in the art, including the following aspects. The siRNA consists of two strands of ribonucleotides which hybridize along a complementary region under physiological conditions. The strands are separate but they may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

The siRNA molecules in accordance with the present invention comprise a double-stranded region which is substantially identical to a region of the mRNA of the target gene. A region with 100% identity to the corresponding sequence of the target gene is suitable. This state is referred to as “fully complementary.” However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

Another factor affecting the efficiency of the RNAi reagent is the target region of the target gene. The region of a target gene effective for inhibition by the RNAi reagent may be determined by experimentation. A suitable mRNA target region would be the coding region. Also suitable are untranslated regions, such as the 5′-UTR, the 3′-UTR, and splice junctions. For instance, transfection assays as described in Elbashir S. M. et al, 2001 EMBO J., 20, 6877-6888 may be performed for this purpose. A number of other suitable assays and methods exist in the art which are well known to the skilled person.

The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer.

Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides.

The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention confer a high in vivo stability suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918 and will not be repeated here. Suitable modifications for oral delivery are more specifically set out in the Examples and description herein. Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides with for instance phosphorothioates, chiral phosphorothioates or phosphorodithioates). Finally, end modifications sometimes referred to herein as 3′ caps or 5′ caps may be of significance. Caps may consist of more complex chemistries which are known to those skilled in the art.

In one embodiment, the invention provides double-stranded ribonucleic acid (dsRNA) molecules for inhibiting the expression of the MAPKAP gene. The dsRNA comprises at least two sequences that are complementary to each other. The dsRNA comprises a sense strand comprising a first sequence and an antisense strand comprising a second sequence. The antisense strand comprises a nucleotide sequence which is substantially complementary to at least part of an mRNA encoding MAPKAP, and the region of complementarity is less than 30 nucleotides in length, generally 19-24 nucleotides in length. The dsRNA, upon contacting with a cell expressing the MAPKAP, inhibits the expression of the MAPKAP gene by at least 40%.

In another embodiment, the invention provides a cell comprising one of the dsRNAs of the invention. The cell is generally a mammalian cell, such as a human cell.

In another embodiment, the invention provides a pharmaceutical composition for inhibiting the expression of the MAPKAP gene in an organism, generally a human subject, comprising one or more of the dsRNA of the invention and a pharmaceutically acceptable carrier or delivery vehicle.

In another embodiment, the invention provides a method for inhibiting the expression of the MAPKAP gene in a cell, comprising the following steps:

(a) introducing into the cell a double-stranded ribonucleic acid (dsRNA), wherein the dsRNA comprises at least two sequences that are complementary to each other. The dsRNA comprises a sense strand comprising a first sequence and an antisense strand comprising a second sequence. The antisense strand comprises a region of complementarity which is substantially complementary to at least a part of a mRNA encoding MAPKAP, and wherein the region of complementarity is less than 30 nucleotides in length, generally 19-24 nucleotides in length, and wherein the dsRNA, upon contact with a cell expressing the MAPKAP, inhibits expression of the MAPKAP gene by at least 40%; and

(b) maintaining the cell produced in step (a) for a time sufficient to obtain degradation of the mRNA transcript of the MAPKAP gene, thereby inhibiting expression of the MAPKAP gene in the cell.

In another embodiment, the invention provides methods for treating, preventing or managing pathological processes mediated by PI3K/AKT/mTOR-signaling, e.g. cancers, comprising administering to a patient in need of such treatment, prevention or management a therapeutically or prophylactically effective amount of one or more of the siRNAs of the invention.

In another embodiment, the invention provides vectors for inhibiting the expression of the MAPKAP gene in a cell, comprising a regulatory sequence operably linked to a nucleotide sequence that encodes at least one strand of one of the siRNA of the invention.

Inhibitory nucleic acid compounds of the present invention may be synthesized by conventional means on a commercially available automated DNA synthesizer, e.g. an Applied Biosystems (Foster City, Calif.) model 380B, 392 or 394 DNA/RNA synthesizer, or like instrument. Phosphoramidite chemistry may be employed. The inhibitory nucleic acid compounds of the present invention may also be modified, for instance, nuclease resistant backbones such as e.g., phosphorothioate, phosphorodithioate, phosphoramidate, or the like, described in many references may be used. The length of the inhibitory nucleic acid has to be sufficient to ensure that the biological activity is inhibited. Thus, for instance in case of antisense oligonucleotides, has to be sufficiently large to ensure that specific binding will take place only at the desired target polynucleotide and not at other fortuitous sites. The upper range of the length is determined by several factors, including the inconvenience and expense of synthesizing and purifying oligomers greater than about 30-40 nucleotides in length, the greater tolerance of longer oligonucleotides for mismatches than shorter oligonucleotides, and the like. Preferably, the antisense oligonucleotides of the invention have lengths in the range of about 15 to 40 nucleotides. More preferably, the oligonucleotide moieties have lengths in the range of about 18 to 25 nucleotides.

Double-stranded RNA, i.e., sense-antisense RNA, also termed small interfering RNA (siRNA) molecules, can also be used to inhibit the expression of nucleic acids for MAPKAP. RNA interference is a method in which exogenous, short RNA duplexes are administered where one strand corresponds to the coding region of the target mRNA (Elbashir et al. (2001) Nature 411: 494). Upon entry into cells, siRNA molecules cause not only degradation of the exogenous RNA duplexes, but also of single-stranded RNAs having identical sequences, including endogenous messenger RNAs. Accordingly, siRNA may be more potent and effective than traditional antisense RNA methodologies since the technique is believed to act through a catalytic mechanism. Preferred siRNA molecules are typically from 19 to 25 nucleotides long, preferably about 21 nucleotides in length and comprise the sequence of a nucleic acid for E2-EPF5. Effective strategies for delivering siRNA to target cells include, for example, transduction using physical or chemical transfection. Alternatively siRNAs may be expressed in cells using, e.g., various PolIII promoter expression cassettes that allow transcription of functional siRNA or precursors thereof. See, for example, Scherr et al. (2003) Curr. Med. Chem. 10(3):245; Turki et al. (2002) Hum. Gene Ther. 13(18):2197; Cornell et al. (2003) Nat. Struct. Biol. 10(2):91. The invention also covers other small RNAs capable of mediating RNA interference (RNAI) such as for instance micro-RNA (miRNA) and short hairpin RNA (shRNA).

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Binders of mTOR/AKT/PI3K Pathway

In order to identify novel modulators of mTOR/AKT/PI3K pathway, a systematic tandem affinity purification (TAP) method was applied to several known signaling molecules of the mTOR pathway. As described in Rigaut et al. (Nat Biotechnol. (1999) 17(10): 1030) and Bouwmeester et al. (Nature Cell Biology (2004) 6: 97-105), the contents of which are hereby incorporated by reference, the TAP purification method involves the fusion of the TAP tag to the target protein of interest and the introduction of the construct into the cognate host cell or organism.

The TAP tag is a tandem fusion of (i) IgG-binding units of protein A from Staphylococcus aureus (ProtA); and (ii) the Calmodulin Binding Peptide (CBP), separated by a TEV protease cleavage site. It allows the rapid purification of complexes from a relatively small number of cells without prior knowledge of the complex composition, activity, or function. Combined with mass spectrometry, the TAP strategy allows for the identification of proteins interacting with a given target protein

Purification of protein complexes from HeLa cells A vector encoding a fusion of hVPS34 to the CBP-TEV-Protein A double tag was constructed using standard methods. Retrovirus encoding the hVPS34 fusion transgene was prepared and used to transducer HeLa cells. Proteins were extracted from the cultured cells using NP-40 detergent lysis buffer. The complex was purified by binding to IgG-linked beads, eluting by TEV protease cleavage, binding of the eluted material on calmodulin containing beads followed by elution with EGTA. All steps were carried out at 0-4° C., excepted for TEV cleavage.

Protein samples were separated by SDS-PAGE, complete gel lanes were systematically cut into slices and proteins were digested in-gel with trypsin as described in Shevchenko (Shevchenko, A., Wilm, M., Vorm, O. & Mann, M. Mass spectrometric sequencing of proteins silver-stained polyacrylamide gels. Anal. Chem. 68, 850-858 (1996). Protein identification was performed by LC-MS/MS, and MS data were searched against an in-house curated version of the International Protein Index (IPI), maintained at the EBI (Hinxton, UK). Results of database searches were read into a database system for further bioinformatics analysis.

MAPKAP was found to be associated with mTOR and hVPS34.

### Example 2

### Generation of siRNA Sequences

The siRNA sequence design uses a BIOPREDsi potency predictor algorithm to score 21-mer oligoribonucleotides. Top scoring sequences are examined for theoretical selectivity against a specified transcriptome, according to experimentally defined selectivity criteria siRNAs. In addition, siRNAs were synthesized by Qiagen (Qiagen, Valencia, Calif.) and Dharmacon (Lafayette, Colo.) as 21-nt oligoribonucleotides with a 19 base pair duplex region and two deoxynucleotide overhangs on the 3′-terminus of each strand. The DNA of the sense strand was a dTdT, whereas the overhang of the antisense strand was complementary to the target mRNA.

The siRNA sequences generated are as follows:

| | | |
|---|---|---|
| siRNA Name | Sequence | SEQ ID NO: |

| | | |
|---|---|---|
| MAPKAP 1_1_1 | cgCGAGAACAGTTCAAGGGCA | 1 |
| MAPKAP 1_1_2 | ccCTCAGTACCGCCTGGAGAA | 2 |
| MAPKAP 1_2_1 | ccCAGTCACGCAATATTTAAA | 3 |
| MAPKAP 1_2_2 | caGGTAGGTGAGCTCGTGAAA | 4 |

We claim:

1. A method of treating or preventing a PI3K/AKT/mTOR related disorder in a patient, comprising administering to the patient a MAPKAP antagonist.

2. The method of claim 1, wherein said MAPKAP antagonist is an siRNA.

3. The method of claim 1, wherein said MAPKAP antagonist is a small molecule.

4. The method of claim 1, wherein the PI3K/AKT/mTOR-related disorder is a cancer.

5. The method of claim 4, wherein the cancer is associated with the overexpression of amino acid transporters (e.g., LAT1), or which harbor mutations in PTEN, TSC1/2, or PI3KclassA

6. The method of claim 1, wherein the PI3K/AKT/mTOR-related disorder is a neurodegenerative disorder.

7. The method of claim 1, wherein the PI3K/AKT/mTOR-related disorder is a metabolic disorder.

8. The method of claim 1, wherein the PI3K/AKT/mTOR-related disorder is a hamartoma syndrome.

9. The method of claim 1, wherein the PI3K/AKT/mTOR-related disorder is a genetic muscle disorders or myopathy.

10. A method of treating or preventing a PI3K/AKT/mTOR-related disorder in a patient, comprising administering to the patient an agent capable of disrupting the binding event between MAPKAP, mTOR, and hVPS34.

11. A method for identifying agents capable of treating or preventing a PI3K/AKT/mTOR related disorder, comprising:
a) providing i) a mTOR complex member protein or homolog capable of binding to a MAPKAP protein; ii) a MAPKAP protein or homolog known to interact with said mTOR complex member; and iii) one or more test agents for screening;
b) mixing, in any order, said mTOR complex member protein or homolog, said MAPKAP protein or homolog, and said one or more compound to be tested; and
c) measuring the alteration of the mTOR complex member protein: MAPKAP binding in the presence of the test agent, as compared to the binding in absence of said agent.

12. The method of claim 11, wherein said agent is a small molecule.

13. The method of claim 11, wherein said agent is an siRNA.

14. A method for identifying agents capable of treating or preventing a PI3K/AKT/mTOR related disorder, comprising:
a) providing i) a PI3K complex member protein or homolog capable of binding to a MAPKAP protein; ii) a MAPKAP protein or homolog known to interact with said PI3K complex member or homolog; and iii) one or more test agents for screening;
b) mixing, in any order, said PI3K complex member protein or homolog, said MAPKAP protein or homolog, and said one or more compound to be tested; and
c) measuring the alteration of the PI3K complex member protein: MAPKAP binding in the presence of the test agent, as compared to the binding in absence of said agent.

15. The method of claim 14, wherein said agent is a small molecule.

16. The method of claim 14, wherein said agent is an siRNA.

17. The present invention includes a method of identifying a test agent that modulates the interaction between MAPKAP, mTOR, and hVPS34, comprising:
a) providing (i) a MAPKAP protein or homolog capable of binding to mTOR and hVPS34; (ii) an mTOR protein or homolog capable of binding to MAPKAP and hVPS34; (iii) a hVPS34 protein or homolog capable of binding to mTOR and MAPKAP, and (iv) a test agent;
b) mixing, in any order, said MAPKAP, mTOR, and hVPS34 proteins or homologs, and said test agent; and
c) measuring the alteration of the interaction between MAPKAP, mTOR, and hVPS34 in the presence of the test agent, as compared to the interaction in absence of said test agent.

18. The method of claim 17, wherein said agent is a small molecule.

19. The method of claim 17, wherein said agent is an siRNA.

20. A method for screening agents useful for the treatment of PI3K/AKT/mTOR related disorders, comprising
a) providing (i) a MAPKAP protein or homolog capable of binding to mTOR and hVPS34; (ii) an mTOR protein or homolog capable of binding to MAPKAP and hVPS34; (iii) a hVPS34 protein or homolog capable of binding to mTOR and MAPKAP, and (iv) a test agent;
b) mixing, in any order, said MAPKAP, mTOR, and hVPS34 proteins or homologs, and said test agent; and
c) measuring the alteration of the interaction between MAPKAP, mTOR, and hVPS34 in the presence of the test agent, as compared to the interaction in absence of said test agent.

21. The method of claim 20, wherein said agent is a small molecule.

22. The method of claim 20, wherein said agent is an siRNA.
